(19) United States
(12) Patent Application Publication
(10) Pub. No.: US 2024/0110252 A1
(43) Pub. Date: Apr. 4, 2024

(54) Compositions and Kits for Rapid Detection of SARS-CoV-2 and Methods of Production and Use Thereof

(71) Applicant: THE BOARD OF REGENTS FOR OKLAHOMAAGRICULTURAL AND MECHANICAL COLLEGES, Stillwater, OK (US)

(72) Inventors: Francisco Manuel Ochoa Corona, Stillwater, OK (US); Andrea Mercedes SALAZAR AGUIRRE, Stillwater, OK (US)

(21) Appl. No.: 18/264,688

(22) PCT Filed: Feb. 11, 2022
(86) PCT No.: PCT/US2022/016139

§ 371 Date: Aug. 8, 2023

Related U.S. Application Data
(60) Provisional application No. 63/148,690, filed on Feb. 12, 2021.

(51) Int. Cl. C12Q 1/70

(52) U.S. Cl. CPC C12Q 1/701; C12Q 2600/158; C12Q 2600/166

(57) ABSTRACT

Kits, devices, systems, and methods are disclosed for the detection of SARS-CoV-2. The kits, devices, systems, and methods utilize two different pairs of polymerase amplification oligonucleotide primers. The first polymerase amplification oligonucleotide primer pair comprises an isolated forward oligonucleotide primer and an isolated reverse oligonucleotide primer for a portion of a SARS-CoV-2 RNA-dependent RNA polymerase (RdRp) or Helicase (Hel) gene sequence. The second polymerase amplification oligonucleotide primer pair comprises an isolated forward oligonucleotide primer and an isolated reverse oligonucleotide primer for a portion of a SARS-CoV-2 Envelope (E) gene sequence. The kits, devices, systems, and methods can specifically detect SARS-CoV-2 infection as well as infection with another Betacoronavirus.

### CROSS REFERENCE TO RELATED APPLICATIONS/INCORPORATION BY REFERENCE

The subject application is a US National Stage Application under 35 USC § 371 of International Application No. PCT/US2022/16139, filed Feb. 11, 2022; which claims benefit under 35 USC § 119(e) of provisional application U.S. Ser. No. 63/148,690, filed Feb. 12, 2021. The entire contents of the above-referenced patent application(s) are hereby expressly incorporated herein by reference.

### STATEMENT REGARDING FEDERALLY SPONSORED RESEARCH OR DEVELOPMENT

Not applicable.

### REFERENCE TO SEQUENCE LISTING SUBMITTED ELECTRONICALLY

The instant application contains, as a separate part of the present disclosure, a Sequence Listing which has been submitted via EFS-Web in computer readable form as an ASCII plain text file. The Sequence Listing, created Feb. 11, 2022, is named “57910179wo_SequenceListing_02112022.txt” and is 7,191 bytes in size. The entire contents of the Sequence Listing are hereby incorporated herein by reference.

### BACKGROUND

Severe acute respiratory syndrome coronavirus 2 (SARS-CoV-2) is a newly-emergent coronavirus which causes a severe acute respiratory disease, COVID-19 (Coronavirus Disease 2019). Clinical features of COVID-19 include fever, dry cough, and fatigue, and the disease can cause respiratory failure resulting in death. The virus spreads readily from person to person primarily through infected secretions, such as saliva and respiratory droplets or aerosols. Evidence supports spread by both symptomatic and asymptomatic individuals.

Currently, the gold standard for diagnosing SARS-CoV-2 infection is PCR amplification. This diagnostic method requires the dilution of biological sample and extraction of RNA therefrom, followed by an RT-PCR reaction, which requires a thermocycler. Therefore, the current PCR diagnostic method for SARS-CoV-2 infection requires transportation of a biological sample to a laboratory setting, dilution/treatment/extraction of the sample prior to assay, and the use of complex instrumentation.

In particular, the current SARS-CoV-2 RT-PCR detection method uses RNA sourced mainly from nasopharyngeal, oropharyngeal, mid-turbinate swabs collected in 2 mL of either Universal Transport Media (UTM) or Virus Transport Media (VTM), and the RNA is extracted from an aliquot of 400 μL, meaning five times diluted. Although RT-PCR sensitivity is powerful, false negative risks remain for specimens from patients with a low virus titer. Rush of refrigerated transportation from a peripheral point of care to centralized laboratories also adds cost and logistics. Further, most RT-PCR reactions require at least two to three hours to complete after transportation, dilution, and treatment processes are performed.

Therefore, there is a need in the art for new and improved compositions and methods for use in diagnosing SARS-CoV-2 infection. It is to such compositions, kits, devices, and methods that the present disclosure is directed.

### BRIEF DESCRIPTION OF THE DRAWINGS

FIG. 1 depicts a map showing RT-RPA nfo primer and probe sequences in SARS-CoV-2 genomic regions for use in one non-limiting embodiment of an RT-RPA nfo assay constructed in accordance with the present disclosure. RNA dependent RNA polymerase gene sequence, SEQ ID NO:21. Envelope protein gene sequence, SEQ ID NO:22. Sequence identifiers for the primers and probes are shown in Table 1.

FIG. 2 schematically illustrates detection of SARS-CoV-2 infection in a biological sample utilizing one non-limiting embodiment of an RT-RPA nfo assay and assay devices constructed in accordance with the present disclosure. The positive line at C indicates the reaction control, the positive line at 2 indicates detection of SARS-CoV-2 RNA dependent RNA polymerase (RdRp) gene, and the positive line at 1 indicates detection of SARS-CoV-2 Envelope (E) gene.

FIG. 3 schematically illustrates optimal probe concentration RT-RPA nfo assays using an artificial positive control (APC) and performed at 26.5° C. and 36° C. with 0.6 μl probe and 1.5 μl primers. Left, NTC and plasmid artificial positive control (APC) reactions carried out at 26.5° C. Right, APC control and NTC reactions carried out at 36° C.

FIG. 4 illustrates a serial dilution sensitivity assay of the RT-RPA nfo assay using plasmid artificial positive control (APC) diluted in water as the sample. Dilutions correspond to the following amounts of APC: from left to right, 1 ng, 0.1 ng, 0.01 ng, 1 pg, 0.1 pg, 0.01 pg, and 1 fg. NTC, no APC.

FIG. 5 illustrates a serial dilution sensitivity assay of the RT-RPA nfo assay using plasmid artificial positive control (APC) diluted in viral transport medium (VTM) as the sample. Dilutions correspond to the following amounts of APC: from left to right, 1 ng, 0.1 ng, 0.01 ng, 1 pg, 0.1 pg, 0.01 pg, and 1 fg. NTC, no APC.

FIG. 6 illustrates a serial dilution sensitivity assay of the RT-RPA nfo assay using Twist Synthetic SARS-CoV-2 RNA Control 2 (Wuhan) diluted in water as the sample. Dilutions correspond to the following amounts of Control 2: from left to right, 1 ng, 0.1 ng, 0.01 ng, 1 pg, 0.1 pg, 0.01 pg, and 1 fg. APC, positive control with APC as sample. NTC, no APC.

FIG. 7 illustrates a specificity assay of the RT-RPA nfo assay using: left, a respiratory viruses control (which contains MERS coronavirus RNA control plus total respiratory viral panel control); middle, a sample of the Delta variant of SARS-CoV-2; and right, NTC, negative control. The specificity assay demonstrates that the assays disclosed herein can specifically detect variants of SARS-CoV-2 and can also detect other coronaviruses and distinguish between those infections and SARS-CoV-2 infection.

FIG. 8 illustrates a specificity assay of the RT-RPA nfo assay using various synthetic SARS-CoV-2 RNA controls constructed as described herein.

FIG. 9 depicts a map showing RT-RPA primer sequences in SARS-CoV-2 genomic regions for use in one non-limiting embodiment of an RT-RPA Basic assay constructed in accordance with the present disclosure. Helicase gene sequence, SEQ ID NO:23. Envelope protein gene sequence, SEQ ID NO:23. Sequence identifiers for the primers and probes are shown in Table 2.

FIG. 10 illustrates a serial dilution sensitivity assay of the RT-RPA Basic assay using Twist Synthetic SARS-CoV-2 RNA Control 2 (Wuhan) diluted in water as the sample. Dilutions correspond to the following amounts of Control 2: from left to right, 1 ng, 0.1 ng, 0.01 ng, 1 pg, 0.1 pg, 0.01 pg, and 1 fg. NTC, negative control.

FIG. 11 illustrates a serial dilution sensitivity assay of the RT-RPA Basic assay using Twist Synthetic SARS-CoV-2 RNA Control 2 (Wuhan) diluted in VTM as the sample. Dilutions correspond to the following amounts of Control 2: from left to right, 1 ng, 0.1 ng, 0.01 ng, 1 pg, 0.1 pg, 0.01 pg, and 1 fg. NTC, negative control.

FIG. 12 illustrates a specificity assay of the RT-RPA Basic assay using: left, a respiratory viruses control (which contains MERS coronavirus RNA control plus total respiratory viral panel control); middle, a sample of the Delta variant of SARS-CoV-2; and right, NTC, negative control. The specificity assay demonstrates that the assays disclosed herein can specifically detect variants of SARS-CoV-2 and can also detect other coronaviruses and distinguish between those infections and SARS-CoV-2 infection.

FIG. 13 illustrates a specificity assay of the RT-RPA Basic assay using various synthetic SARS-CoV-2 RNA controls constructed as described herein.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

Turning now to the non-limiting embodiments of the present disclosure, provided herein are kits, devices, systems, and methods for detecting SARS-CoV-2. The kits, devices, systems, and methods rely on amplification primers selected based on a specific portion of the SARS-CoV-2 RNA-dependent RNA polymerase (RdRp) or Helicase (Hel) gene sequence (that is specific for SARS-CoV-2) and a specific portion of the SARS-CoV-2 Envelope (E) gene sequence (that is conserved among betacoronaviruses). In certain particular (but non-limiting) embodiments, the kits, devices, systems, and methods rely on recombinase polymerase amplification (RPA) as an easy-to-use and rapid isothermal detection method. Alternatively, the kits, devices, systems, and methods of the present disclosure can be utilized with other polymerase amplification techniques, such as (but not limited to) polymerase chain reaction (PCR).

Certain non-limiting embodiments of the present disclosure are directed to a kit containing a plurality of polymerase amplification (such as, but not limited to, recombinase polymerase amplification (RPA) or PCR) oligonucleotide primer pairs, wherein each pair of oligonucleotide primers comprises an isolated forward oligonucleotide primer and an isolated reverse oligonucleotide primer. In certain non-limiting embodiments, a first polymerase amplification oligonucleotide primer pair comprises an isolated forward oligonucleotide primer and an isolated reverse oligonucleotide primer for a portion of a SARS-CoV-2 RNA-dependent RNA polymerase (RdRp) or Helicase (Hel) gene sequence, while a second polymerase amplification oligonucleotide pair comprises an isolated forward oligonucleotide primer and an isolated reverse oligonucleotide primer for a portion of a SARS-CoV-2 envelope (E) gene sequence. Each of the oligonucleotide primers of the kit comprises adenine (A), cytosine (C), guanine (G), and thymine (T) residues; that is, the oligonucleotide primers of the kit differ from the native SARS-CoV-2 RNA sequences via the presence of thymine (T) residues instead of uracil (U) residues.

In certain particular (but non-limiting) embodiments, the reverse primer for each oligonucleotide pair is conjugated to a chemical or biochemical capture moiety for diagnostic applications, wherein the capture moiety is one member of a specific binding pair (while the other member of the specific binding pair is immobilized on a surface or other form of substrate so as to “capture” amplification products that contain the capture moiety). Non-limiting examples of moieties to which the reverse primers can be conjugated include biotin, Biosg, streptavidin, avidin, traptavidin, colloidal carbon in the form of ‘India ink’, collodial gold, fluorescent molecules, quantum dots nanocrystals, semiconductor nanocrystals, upconverting phosphors, bioluminescent markers, luminescent oxygen channeling, enzyme labels, paramagnetic particles, latex particles, and the like, as well as any combinations thereof.

The oligos of the oligonucleotide primer pairs may correspond to any portion of the SARS-CoV-2 RdRp/Hel and E gene sequences (or a variant or derivative thereof) and may have any structural characteristics or modifications known in the art, so long as the oligonucleotide pairs are able to function in accordance with the present disclosure.

For example (but not by way of limitation), each oligonucleotide of the oligonucleotide pair(s) may be provided with any length that allows the oligonucleotides to amplify the corresponding template of the SARS-CoV-2 gene sequence and function in accordance with the present disclosure. Non-limiting examples of lengths that may be utilized in accordance with the present disclosure include about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70 nucleotides, and larger or smaller lengths. The scope of the present disclosure also explicitly includes ranges of lengths formed of two of any of the above values (i.e., a range of from about 30 nucleotides to about 45 nucleotides, etc.).

In addition, each oligonucleotide of the RPA oligonucleotide pair(s) may be provided with any G/C content that allows the oligonucleotide pair to amplify the corresponding template of the SARS-CoV-2 gene sequence and function in accordance with the present disclosure. Non-limiting examples of G/C contents that may be utilized in accordance with the present disclosure include about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, and about 70%, as well as higher or lower G/C contents. The scope of the present disclosure also explicitly includes ranges of G/C contents formed of two of any of the above values (i.e., a range of from about 40% to about 60%, etc.).

In addition, each oligonucleotide of the RPA oligonucleotide pair(s) may be provided with any Tm that allows the oligonucleotide pair to amplify the corresponding template of the SARS-CoV-2 gene sequence and function in accordance with the present disclosure. Non-limiting examples of Tm's that may be utilized in accordance with the present disclosure include about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., and about 110° C., as well as higher or lower Tm's. The scope of the present disclosure also explicitly includes ranges of Tm's formed of two of any of the above values (i.e., a range of from about 50° C. to about 100° C., etc.).

In certain non-limiting embodiments, the forward oligonucleotide primers may each contain a DNA sequence that is homologous to a portion of a sequence of any of the SARS-CoV-2 RdRp, Hel, or E gene sequences known in the art or disclosed or otherwise contemplated herein, while the reverse oligonucleotide primers may each have a DNA sequence that is complementary to a portion of a sequence selected from the RdRp, Hel, or E sequences.

In certain particular (but non-limiting) embodiments, the kit further contains an isolated oligonucleotide probe for detection of the RdRp or Hel gene sequence and an isolated oligonucleotide probe for detection of the E gene sequence.

In certain particular (but non-limiting) embodiments, at least one oligonucleotide from each oligonucleotide pair, or a probe utilized with an oligonucleotide pair (when present), includes a label for detection of the amplification product. In addition, the label utilized with the RdRp/Hel primers/probe differs from the label utilized with the E primers/probe. A wide variety of nucleic acid labels useful in detecting an amplification product are well known in the art, and numerous nucleic acid labels are commercially available. Non-limiting examples of nucleic acid labels that may be utilized in accordance with the present disclosure include a fluorescein (such as, but not limited to, fluorescein amidite, fluorescein isothiocyanate (FITC), or carboxyfluorescein), digoxin, digoxigenin, Cy5, Texas Red, 2,4-dinitrophenyl (DNP), fluorescent molecules, quantum dots nanocrystals, semiconductor nanocrystals, upconverting phosphors, bioluminescent markers, luminescent oxygen channeling, enzyme labels, paramagnetic particles, latex particles, and the like.

In certain particular (but non-limiting) embodiments, the RPA oligonucleotide primer pairs and probes comprise sequences as represented in Table 1.

TABLE 1
Oligonucleotide Primers and Probes for SARS-COV-2 RdRp and E Genes

| Name | Sequence | SEQ ID NO: |
|---|---|---|
| RdRp Forward | TGGAACCTCATCAGGAGATGCCACAACTGCTTATG | 1 |
| RdRp Reverse | GAGACACTCATAAAGTCTGTGTTGTAAATTGCGGA | 2 |
| RdRp Probe | GTGTTTTTAACATTTGTCAAGCTGTCACGGCCATGTTAATGCACTTT | 3 |
| E Forward | GTACTCATTCGTTTCGGAAGAGACAGGTACGTTAA | 4 |
| E Reverse | AAACGTAAAAAGAAGGTTTTACAAGACTCACG | 5 |
| E Probe | TTTCGTGGTATTCTTGCTAGTTACACTAGCCATCCTTATGCGCTTCGATTGTGT | 6 |

In a particular (but non-limiting) embodiment, the RPA oligonucleotide primers and probes comprise the sequences of SEQ ID NOS:11-16, as outlined in Table 3 in Example 1 below.

In other particular (but non-limiting) embodiments, the RPA oligonucleotide primer pairs comprise sequences as represented in Table 2.

TABLE 2
Oligonucleotide Primers for SARS-COV-2
Hel and E Genes

| | | SEQ |
|---|---|---|
| Name | Sequence | ID NO: |
| Hel Forward | CATAAATTAGTCTTGTCTGTTAATCCGTAT | 7 |
| | GTTTG | |
| Hel Reverse | GTGATTTACAATAATAGCTCATACCTCCTA | 8 |
| | AGTAA | |
| E Forward | GGAAGAGACAGGTACGTTAATAGTTAATAG | 9 |
| E Reverse | AAAAGAAGGTTTTACAAGACTCACGTTAAC | 10 |

In a particular (but non-limiting) embodiment, the RPA oligonucleotide primers comprise the sequences of SEQ ID NOS:17-20, as outlined in Table 5 in Example 2 below.

While SEQ ID NOS:1-20 are shown herein for exemplary purposes, it will be understood that the scope of the present disclosure is not limited to the particular sequences shown in the Tables. That is, a person having ordinary skill in the art will readily understand that the lengths of each sequence may be altered slightly and still be capable of amplifying the gene sequences in the manner disclosed herein. For example (but not by way of limitation), each of the oligonucleotide primers utilized in accordance with the present disclosure may differ from or be shorter than the primer of any of SEQ ID NOS:1-20 by about five residues or less, about four residues or less, about three residues or less, about two residues or less, or about one residue. In addition, each of the oligonucleotide primers utilized in accordance with the present disclosure may differ from the primer of any of SEQ ID NOS:1-20 by addition of one or more residues on a 5′ and/or 3′ end thereof, such as, but not limited to, about one residue, about two residues, about three residues, about four residues, about five residues, about six residues, about seven residues, about eight residues, about nine residues, about ten residues, or more. These added sequences may correspond to the corresponding gene sequence, or a portion of these added sequences may differ from the corresponding gene sequence by one or more residues (such as, for example but not by way of limitation, to aid in attachment of a capture moiety or label thereto).

Each of the oligonucleotides of an oligonucleotide pair may function as a primer and/or a probe, where the primer primes the synthesis of the reaction with the assistance of the pool of replicase-enzymes to generate millions of copies of the targeted region, while the probe labels each of these newly produced copies in a specific internal region to generate additive chemistry to each new copy generated, thereby allowing a sensitive detection proportional to the number of copies amplified. As such, when an oligonucleotide functions as a probe, the oligonucleotide may be modified, such as (but not limited to) by addition of a label, for detection of the amplification product. One or both of the oligonucleotides of each oligonucleotide pair may contain other modifications as necessary for improving the stability and/or yield of the assay and/or for improving the detection of the assay products. Such modifications to oligonucleotides for functioning as primers or probes are well known in the art, and thus no further discussion thereof is deemed necessary.

The amplification products generated by each of the oligonucleotide pair(s) may be provided with any length that is acceptable under the parameters of the amplification reaction utilized (such as, but not limited to, RPA or PCR) and that can be detected in accordance with the methods of the present disclosure. Non-limiting examples of product lengths that may be utilized in accordance with the present disclosure include about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 310, about 320, about 330, about 340, about 350, about 360, about 370, about 380, about 390, and about 400 base pairs, and larger or smaller base pair lengths. The scope of the present disclosure also explicitly includes ranges of lengths formed of two of any of the above values (i.e., a range of from about 60 base pairs to about 300 base pairs, a range of from about 100 base pairs to about 250 base pairs, etc.).

The kits of the present disclosure may be provided with additional reagents that are used in the amplification reactions and/or detection assays. For example, but not by way of limitation, the kits may include one or more reagents such as (but not limited to), a recombinase, a polymerase, a single-stranded DNA binding protein, betaine, magnesium acetate, rehydration buffer, nuclease-free water, reaction buffers, and combinations thereof.

Also, in certain particular (but non-limiting) embodiments, the kits of the present disclosure may be provided with one or more additional oligonucleotide pairs for conducting another amplification reaction. For example (but not by way of limitation), this additional oligonucleotide primer pair may be for at least one positive or negative control. Non-limiting examples of a positive control that can be utilized with the kits of the present disclosure includes human-defensin 2 (HBD-2).

Further, the kits of the present disclosure may include other positive or negative controls, as known in the art. For example (but not by way of limitation), the kit may include an artificial positive control, as described in detail herein after.

In certain particular (but non-limiting) embodiments, the kit contains: (i) an isolated forward RPA oligonucleotide primer, an isolated reverse RPA oligonucleotide primer, and an isolated probe for detection of the SARS-CoV-2 E gene sequence; (ii) an isolated forward RPA oligonucleotide primer, an isolated reverse RPA oligonucleotide primer, and an isolated probe for detection of the SARS-CoV-2 RdRp or Hel gene sequence; and (iii) an artificial positive control that contains targets for RdRp/Hel and E genes of SARS-CoV-2.

In addition, the kits of the present disclosure may be provided with one or more components used in the amplification reactions and/or detection assays. For example, but not by way of limitation, the kits may include one or more of the following components: (i) at least one reaction chamber for performing a polymerase amplification reaction; (ii) at least one nucleic acid lateral flow assay device; (iii) at least one sample collection device; and/or (iv) at least one nasopharyngeal swab.

Any reaction chambers known in the art as applicable for use with amplification reactions (such as, but not limited to, RPA and PCR) may be utilized in accordance with the present disclosure. The reaction chambers may include one or more dried reagents for performing the amplification reactions. For example (but not by way of limitation), when the reaction is an RPA reaction, the reaction chambers may comprise a dried reagent composition that includes a recombinase, a polymerase, and a single-stranded DNA binding protein. Non-limiting examples of reaction chambers that may be utilized in accordance with the present disclosure include those disclosed in U.S. Pat. Nos. 10,036,057 and 10,538,760 (the entire contents of which are hereby expressly incorporated herein by reference).

Any nucleic acid lateral flow assay devices known in the art for detection of amplification products may be utilized in accordance with the present disclosure. Such devices are well known in the art and commercially available; two non-limiting examples thereof include Milenia HybriDetect lateral flow dipsticks (Milenia Biotec GmbH, GieBen, Germany) and PCRD nucleic acid lateral flow immunoassay (NALFIA) cassettes (Abingdon Health, York, UK). Further non-limiting examples of nucleic acid lateral flow assay devices utilized in combination with RPA reaction chambers are disclosed in U.S. Pat. Nos. 10,036,057 and 10,538,760.

Any sample collection devices known in the art that are amenable for collecting samples for subsequent nucleic acid amplification assays can be utilized in accordance with the present disclosure. In one particular (but non-limiting) embodiment, the sample collection device is an elution independent collection device such as that disclosed in U.S. Pat. No. 9,423,398 (the entire contents of which are hereby expressly incorporated herein by reference). For example (but not by way of limitation), the elution independent collection device may be an apparatus that comprises a backing card, a sample pad adhered to said backing card, a wick adhered to said backing card and spaced apart from said sample pad, and a water-soluble membrane situated between said sample pad and said wick and in communication with both said sample pad and said wick. The water-soluble membrane comprises an affixed portion and an unaffixed portion, said affixed portion being secured to said backing card such that said unaffixed portion is at least large enough to permit a punch to extract a sample therefrom without including any of said affixed portion in the sample, wherein there is a gap between at least a portion of said membrane and said backing card. In addition, said membrane binds an analyte from a sample that flows through a flow path defined by said sample pad, through said membrane to said wick. Also, said unmounted portion of said membrane does not substantially inhibit analysis of the analyte when placed in an analysis system.

Certain non-limiting embodiments of the present disclosure are directed to systems that include various combinations of any of the kit components described herein above or otherwise contemplated herein. For example (but not by way of limitation), certain non-limiting embodiments are directed to a system that includes the two polymerase amplification oligonucleotide primer pairs for SARS-CoV-2 gene sequences as disclosed or otherwise contemplated herein (alone or in combination with the oligonucleotide probes disclosed herein above for use therewith) in combination with one or more of the collection devices disclosed or otherwise contemplated herein, one or more reaction chambers disclosed or otherwise contemplated herein, and/or one or more of any of the nucleic acid lateral flow assay devices disclosed or otherwise contemplated herein. In a particular (but non-limiting) example, each reaction chamber comprises one or more reagents for performing the polymerase amplification reaction.

In a particular (but non-limiting) embodiment, the collection device(s) present in the system may be an elution independent collection device as described in detail herein above.

In a particular (but non-limiting) embodiment, the reaction chamber(s) comprises a reagent composition (such as, but not limited to, a dried reagent composition) comprising a recombinase, a polymerase, and a single-stranded DNA binding protein.

Certain non-limiting embodiments of the present disclosure are directed to a screening method that includes the steps of: obtaining a mammalian sample suspected of containing SARS-CoV-2; combining the mammalian sample with any of the oligonucleotide primer pairs (with or without probes) described or otherwise contemplated herein (including a primer pair for the SARS-CoV-2 E gene sequence and a primer pair for either the SARS-CoV-2 RdRp or the SARS-CoV-2 Hel gene sequence) and an RPA reagent composition to provide a mixture and incubating the mixture under conditions that allow amplification to occur, wherein the RPA reagent composition comprises a recombinase, a polymerase, and a single-stranded DNA binding protein; contacting the incubated mixture with a nucleic acid lateral flow assay device; detecting RdRp/Hel and E RPA products via the nucleic acid lateral flow assay device; determining that SARS-CoV-2 is present in the mammalian sample if RdRp/Hel and E RPA products are detected; and determining that a related coronavirus is present in the mammalian sample if E RPA products are detected and RdRp/Hel RPA products are not detected.

Certain non-limiting embodiments of the present disclosure are directed to a screening method that includes the steps of: obtaining a mammalian sample suspected of containing SARS-CoV-2; combining the mammalian sample with any of the oligonucleotide primer pairs (with or without probes) disclosed or otherwise contemplated herein (including a primer pair for the SARS-CoV-2 E gene sequence and a primer pair for either the SARS-CoV-2 RdRp or the SARS-CoV-2 Hel gene sequence) and a PCR reagent composition to provide a mixture and incubating the mixture under conditions that allow amplification to occur; contacting the incubated mixture with a nucleic acid lateral flow assay device; detecting RdRp/Hel and E PCR products via the nucleic acid lateral flow assay device; determining that SARS-CoV-2 is present in the mammalian sample if RdRp/Hel and E PCR products are detected; and determining that a related coronavirus is present in the mammalian sample if E PCR products are detected and RdRp/Hel PCR products are not detected.

In certain particular (but non-limiting) embodiments of the methods of the present disclosure, a third amplification oligonucleotide primer pair (with or without probe) disclosed or otherwise contemplated herein (such as, but not limited to, an oligonucleotide primer pair associated with a positive control) may be combined with the two other oligonucleotide primer pairs and the mammalian sample.

In certain non-limiting embodiments, the screening method can discriminate between the presence of SARS-CoV-2 and another related respiratory virus, including other betacoronaviruses.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

The following Examples present the detection of SARS-CoV-2 via two methods. In Example 1, the method designated herein as “RT-RPA nfo” is described, which utilizes reverse transcription nfo (Endonuclease IV) replicase polymerase amplification in combination with two primers and one probe for each reaction. In Example 2, the method designated herein as “RT-RPA Basic” is described, which utilizes reverse transcription replicase polymerase amplification with two primers and no probe.

### Example 1

RT-RPA nfo is an isothermal method with a sensibility comparable to PCR based amplification that can be performed at points of care at 37° C. without a thermocycler, and instead with a less costly dry bath. RT-RPA nfo couples isothermal recombinase-driven primers and probes, specifically designed for this purpose and core of this development, with strand-displacement DNA synthesis for amplification of the targeted template.

The diagnostic targets achieve exponential amplification, and there is no need for pretreatment of RNA/DNA specimen. RT-RPA nfo reactions are sensitive, specific, and relatively rapid. Average reactions take approximately one hour from sampling to result (i.e., approximately 20 minutes for RPA reaction and 15 minutes for lateral flow assay). Assay time varies depending on the operator skills and the analyte concentration. RT-RPA nfo is applicable in the detection of so-called sandwich assays, and lateral-flow strip as a readout system. The technology proved to be sensitive to fewer than ten copies. The lateral-flow strip systems of the present disclosure allow for multiple detections of two diagnostic targets simultaneously in combination with a control assay.

The current SARS-CoV-2 RT-PCR detection method uses RNA sourced mainly from nasopharyngeal, oropharyngeal, mid-turbinate swabs collected in 2 mL of either Universal Transport Media (UTM) or Virus Transport Media (VTM), and the RNA is extracted from an aliquot of 400 μL, meaning five times diluted. Although RT-PCR sensitivity is powerful false negative risks remain for specimens from patients with a low virus titer. Rush of refrigerated transportation from a peripheral point of care to centralized laboratories also adds cost and logistics. The elution independent collection device (EICD; U.S. Pat. No. 9,423,398, the entire contents of which are hereby expressly incorporated herein by reference) was developed for the rapid collection of microorganisms and the recovery of nucleic acids. It collects fluid specimens by contact and lateral flow and has been adapted herein to COVID-19 triage and testing. Pieces of a built-in soluble element dissolve directly in commercial RT-RPA nfo and PCR mixtures without an intermediate elution step, thereby streamlining either RT-RPA nfo and PCR based assays.

The assay described in this Example involved the following reactions at the temperatures and times shown: 37° C. for 30 minutes (reverse transcription); 36° C. for 20 minutes (RPA reaction), and 80° C. for 30 minutes (enzyme deactivation). The following volumes of reagents were utilized: 1.05 μl of 10 μM primers; 0.6 μl of 10 μM probe; 1 μl of MMLV-RT; and 2 μl of sample (RNA).

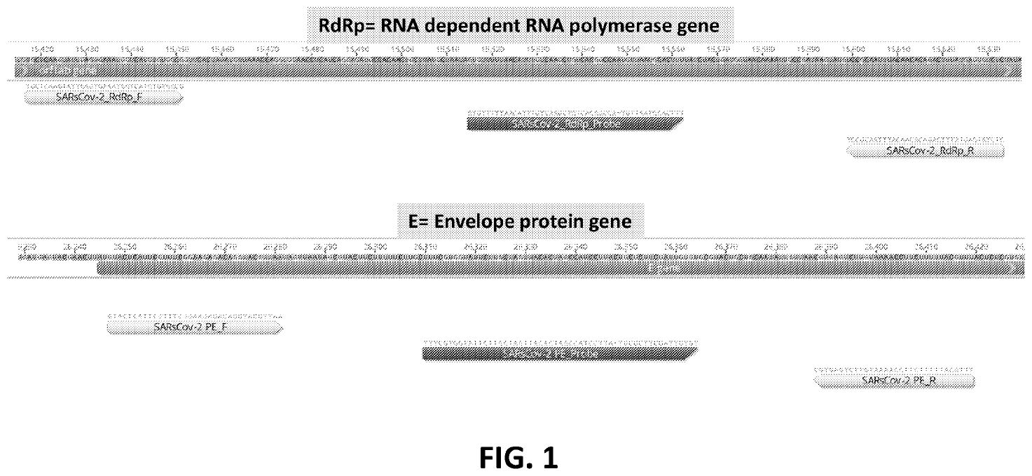

Two SARS-CoV-2 genes were targeted after the alignment of sequences sourced from the NCBI-Genbank: the RdRp and E genes. The first is conserved at the species level, and the second is conserved among Betacoronavirus species. Primer and probe design was made combining software (Primer3 and Primer-BLAST), visually detected virus sequence signatures, and primer design guidelines by the RPA nfo manufacturer TwistDX. The oligonucleotide primers and probes for RPA nfo SARsCov-2_RdRp and E genes including the required chemical labels are presented in Table 3, and FIG. 1 contains a map demonstrating alignment of the primers and probes to the SARS-CoV-2 genomic regions.

TABLE 3
Oligonucleotide Primers and Probes for RPA nfo SARS-COV-2
RdRp and E Genes Including the Required Chemical Labels

| Name | Sequence | SEQ ID NO: |
|---|---|---|
| SARsCov- | TGGAACCTCATCAGGAGATGCCACAACTGCTTATG | 11 |
| 2_RdRp_F | | |
| SARsCov- | [Capture Moiety]- | 12 |
| 2_RdRp_R | GAGACACTCATAAAGTCTGTGTTGTAAATTGCGGA | |
| SARsCov- | [Label 1]-GTGTTTTTAACATTTGTCAAGCTGTCACGGCCA[THF] | 13 |
| 2_RdRp_Probe | TGTTAATGCACTTT-C3spacer | |
| SARsCov-2 PE_F | GTACTCATTCGTTTCGGAAGAGACAGGTACGTTAA | 14 |
| SARsCov-2 PE_R | [Capture Moiety]- | 15 |
| | AAACGTAAAAAGAAGGTTTTACAAGACTCACG | |
| SARsCov-2 | [Label 2]- | 16 |
| PE Probe | TTTCGTGGTATTCTTGCTAGTTACACTAGCCATCCTTA[THF] | |
| | TGCGCTTCGATTGTGT-C3spacer | |

Any capture moieties disclosed or otherwise contemplated herein may be utilized with SEQ
ID NOS: 12 and 15. In the context of this non-limiting example, the capture moiety
utilized was biotin. Label 1 and Label 2 are each selected from any labels disclosed or
otherwise contemplated herein. In the context of this non-limiting example, Label 1 was
FAM, and Label 2 was DIG.

The Conserved CORE-region found in SARs_Cov-2 RdRp contains Primers and Probe located between nt 15432-15599 (190 bp) of the reference accession NC_045512.2. The SARS-Cov-2 reference sequences used to generate a RdRp consensus sequence for RPA primers/probes design are listed in Table 4. The Conserved CORE-region found in SARs_Cov-2 E protein contains Primers and Probe located between nt 15432-15599 (150 bp) of the reference accession NC_045512.2. The SARS-Cov-2 reference sequences used to generate an E consensus sequence for RPA primers/probes design are also listed in Table 4.

TABLE 4
Accession Numbers for Reference Sequences used to Generate
a Consensus Sequence for RPA Primers/Probes

| | |
|---|---|
| SARS-CoV-2 RdRp | SARS-CoV-2 E |
| Protein Sequences | Protein Sequences |

| | | | |
|---|---|---|---|
| LC522350.1 | MT042777.1 | MT127115.1 | MT192765.1 |
| MN938385.1 | MT042778.1 | MT049951.1 | NC_045512.2 |
| MN938386.1 | MT050414.1 | MT093571.1 | MT192773.1 |
| MN970003.1 | MT050415.1 | MT126808.1 | MT192772.1 |
| MN970004.1 | MT050416.1 | MT066176.1 | MT123292.2 |
| MN975263.1 | MT050417.1 | MT066175.1 | MT123291.2 |
| MN975264.1 | MT066157.1 | MT066156.1 | MT152824.1 |
| MN975265.1 | MT066158.1 | LC528233.1 | MT093631.2 |
| MT042773.1 | MT066159.1 | LC528232.1 | |
| MT042774.1 | MT072668.1 | | |
| MT042775.1 | MT127116.1 | | |
| MT042776.1 | MT159778.1 | | |

In this Example, a prototype of RT-RPA nfo was developed for the detection of the severe acute respiratory syndrome coronavirus 2 (SARS-CoV-2). RT-RPA nfo is an isothermal method with a sensibility comparable to PCR based amplification that can be performed at points of care at 37° C. without thermocycler, but a less costly dry bath. The assay includes two major steps: the first is the in vitro wet stage, in which the RPA nfo reaction occurs at 36° C., and a second solid phase stage in which the RPA nfo reaction is transferred into a nucleic acid lateral flow immunoassay (NALFIA), suitable for visual (qualitative) detection of the generated amplification products. The in vitro wet stage was performed using the commercially available TwistAmp® nfo DNA amplication kit (TwistDx™ Limited, Cambridge, UK) using the newly developed oligonucleotide primers presented in Table 2. The second stage was performed in a commercially available PCRD NALFIA from Abingdon Health's or alternative provider designed to simultaneously detect two amplicons labeled with DIG/Biotin and/or FITC (or FAM)/Biotin.

Results were available in approximately one hour, making the assay a convenient solution for rapid detection of SARS-CoV-2 and Betacoronavirus nucleic acids at point of care testing facilities without the need of thermocycler investment. Moreover, there is no need for RNA extraction, and the specimen consists of microliters of collection-captured VTM media.

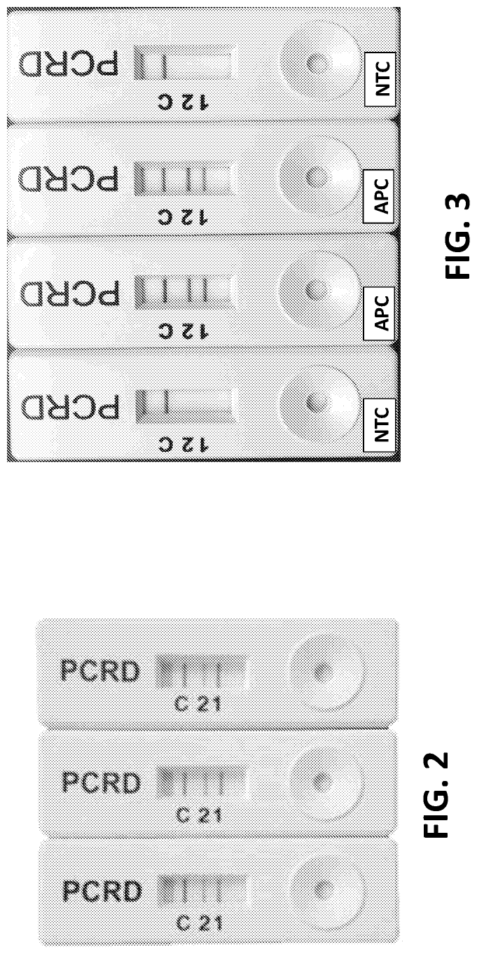

The presented RT-RPA assay detected two SARS-CoV-2 genes targeted after alignment of sequences sourced from the NCBI-Genbank: these are the RdRp and E genes. The first is conserved at the species level, and the second is conserved among Betacoronavirus species. That is, as shown in FIG. 2, Line C is the assay control that assures the test worked well. Line 1 detects the E sequence, which is conserved across many Betacoronavirus species, and line 2 detects the RdRp sequence, which is specific to SARS-CoV-2, the causal agent of COVID-19. An assay positive to Line C only means the patient is SARS-CoV-2 negative. An assay positive to Lines C and 1, but not line 2, means the specimen is Betacoronavirus positive but SARS-CoV-2 negative, and therefore is infected by another related respiratory virus that is not SARS-CoV-2. An assay positive to Lines C, 1, and 2 means the specimen is SARS-CoV-2 positive. An assay negative to line C is inconclusive and requires repetition.

FIG. 3 demonstrates an optimal probe concentration assay using a plasmid artificial positive control (APC) carrying RdRp and E gene targets of SARS-CoV-2 in the RT-RPA nfo assays performed at 26.5° C. and 36° C. with 0.6 μl probe and 1.5 μl primers. The optimal probe concentration and temperature 0.6 μl at 26.5° C. (left) is flexible and allows positive reaction as seen at 1.5 μl at 36° C. (right). Non-Template Controls (NTC) were included (far left and right).

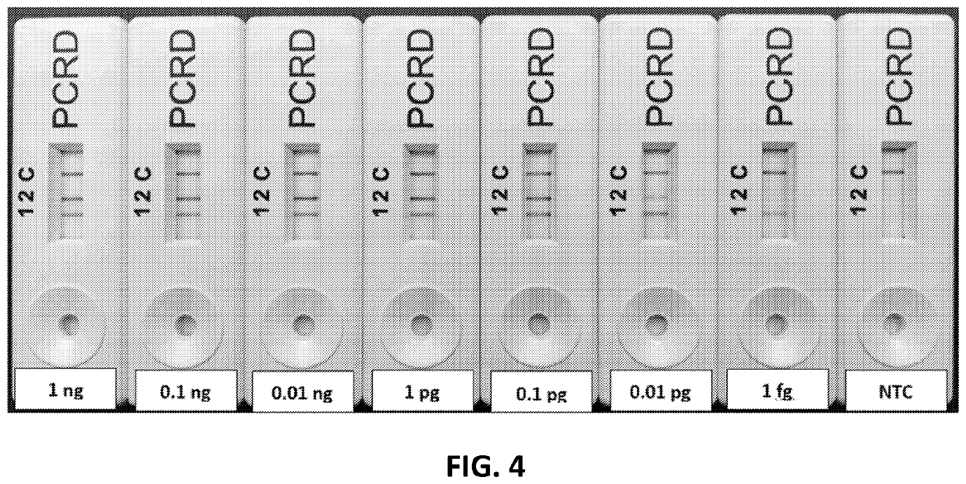

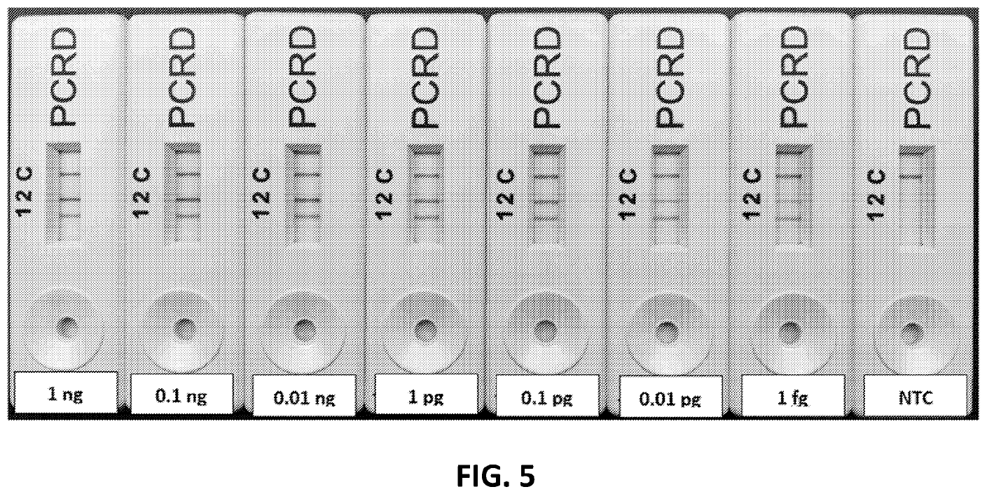

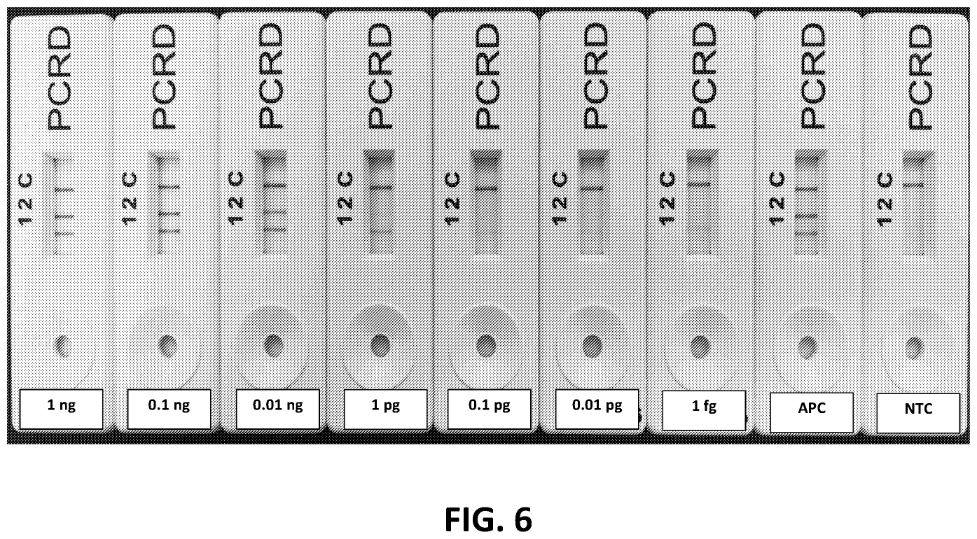

FIGS. 4-5 illustrate serial dilution limit of detection assays of the RT-RPA nfo method using plasmid artificial positive control (APC) diluted in water (FIG. 4) or virus transport medium (VTM; FIG. 5) as the sample. As can be seen, the APC could be detected at the lowest concentration tested (1 fg). FIG. 6 demonstrates another limit of detection of the RT-RPA nfo method using Synthetic SARS-CoV-2 RNA Control 2 (Wuhan) diluted in water as the sample. As can be seen, SARS-CoV-2 RdRp RNA of the synthetic control was also detected at a concentration as low as 1 pg. The SARS-CoV-2 RNA of E was detected at a concentration of 1 fg.

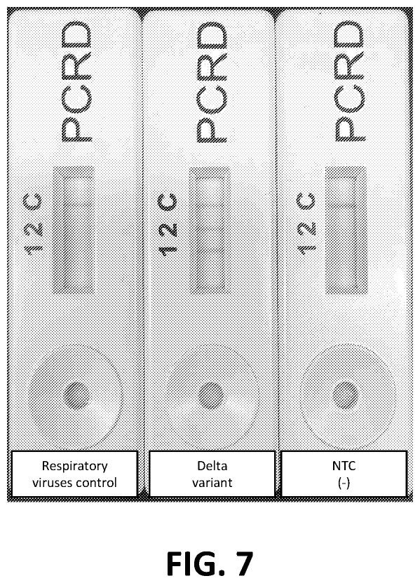

FIG. 7 demonstrates the specificity of the RT-nfo-RPA assay. Left, the assay did not amplify (positive reaction) products from either the total respiratory viral panel control (swab) combined with the MERS Coronavirus RNA control. Center, positive reaction of the RdRp and E genes from the synthetic SARS-CoV-2 Delta variant RNA Artificial Positive Control. Right, NTC included as the negative control.

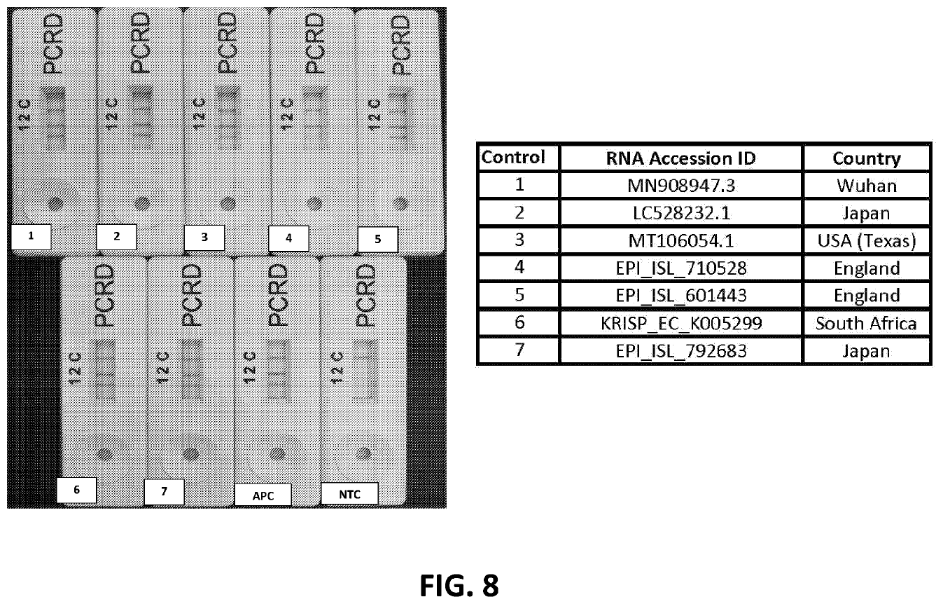

FIG. 8 illustrates a specificity assay of the RT-RPA nfo assay using various synthetic SARS-CoV-2 RNA controls. Synthetic viral controls such as those described herein are a powerful alternative to “live virus” controls which are viral nucleic acids extracted from either an infected patient or from infectious virus propagated in cell culture. Synthetic controls created through gene synthesis broaden access across diverse strains while mitigating safety and security concerns. At present, there are thousands of variants of the SARS-CoV-2 virus available in public repositories.

The synthetic SARS-CoV-2 RNA controls, seven total, include six non-overlapping 5 kb fragments generated from gene fragments then transcribed into ssRNA, and provide coverage of 99.9% of the bases of the viral genome. The GenBank IDs and GISAID Name for synthetic controls 1 through 7 are shown in FIG. 8. These synthetic RNA controls serve as sequence diverse position controls mimicking diversity found globally as this virus evolves. As can be seen in FIG. 8, these synthetic controls were effective in demonstrating the specificity of the assay.

In addition, in certain non-limiting embodiments, the present disclosure incorporates the SARS-CoV-2 RT-RPA nfo assay with an elution independent collection device (such as, but not limited to, the device described in U.S. Pat. No. 9,423,398 and developed for the rapid collection of microorganisms and the recovery of nucleic acids). This collection device collects fluid specimens by contact and lateral flow and can be adapted to COVID-19 triage and testing. Pieces of a built-in soluble element dissolve directly in commercial RT-RPA nfo and PCR mixtures without an intermediate elution step, thereby streamlining either RT-RPA nfo and PCR based assays and facilitating storage at room temperature of the specimens.

COVID-19 Assay Kit

In this particular (but non-limiting) Example, a COVID-19 assay kit contains: i) A collection-capture method to improve virus yields at sampling consisting of collection tubes with reduced volume (500 μL) of VTM media (or water or PBS buffer) and nasopharyngeal swabs, ii) Elution independent collection devices and buffer for sample preservation or shipping, and iii) a formulation of lyophilized primers and probes for use in commercial SARS-CoV-2 RPA nfo reactions compatible with PCRD NALFIA for rapid and sensitive visualization of the assay result. The last two commercially available. The COVID-19 assay kit is an innovative and attractive collection, detection, and diagnostic technological package for one-hour processing of severe acute respiratory syndrome coronavirus 2 (SARS-CoV-2) specimens.

### Example 2

In this Example, another non-limiting embodiment of compositions and methods utilized in the detection of SARS-CoV-2 are described. This method is designated herein as “RT-RPA Basic” and is similar to the RT-RPA nfo method described in Example 1 and utilizes reverse transcription replicase polymerase amplification with two primers. However, this method differs from the RT-RPA nfo method in that no probes are utilized. Rather, the two labels that were attached to the probes in the RT-RPA nfo method are instead attached to the forward oligonucleotide primers. In addition, while the RT-RPA nfo method amplified a portion of the SARS-CoV-2 RdRp gene sequence for specific detection of SARS-CoV-2, the RT-RPA Basic method instead amplifies a portion of the SARS-CoV-2 Helicase (Hel) gene sequence for specific detection of SARS-CoV-2.

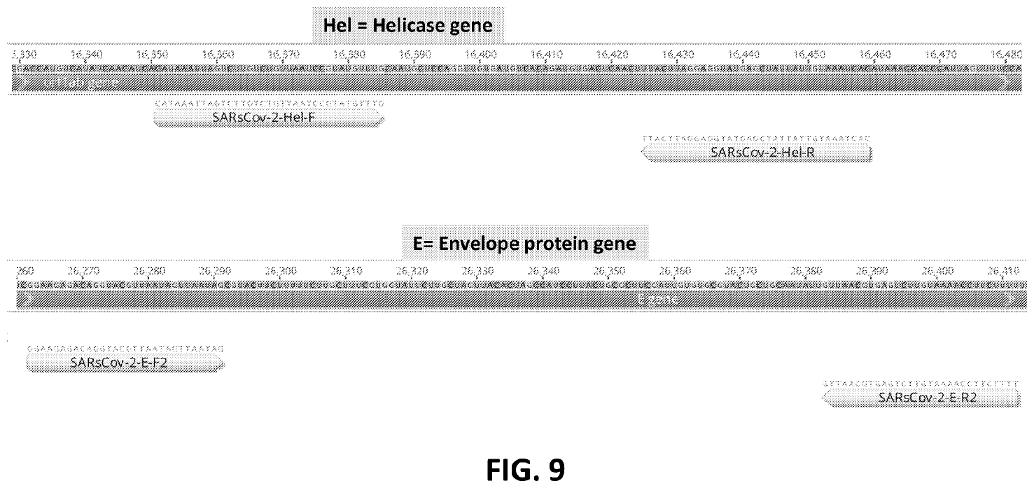

The oligonucleotide primers and probes for RT-RPA Basic SARS-CoV-2 Hel and E genes including the required chemical labels are presented in Table 5, and FIG. 9 contains a map demonstrating alignment of the primers and probes to the SARS-CoV-2 genomic regions.

TABLE 5
Oligonucleotide Primers for SARS-COV-2 Hel and E Genes

| Name | Sequence | SEQ ID NO: |
|---|---|---|
| Hel Forward | [Label 1]-CATAAATTAGTCTTGTCTGTTAATCCGTATGTTTG | 17 |
| Hel Reverse | [Capture Moiety]-GTGATTTACAATAATAGCTCATACCTCCTAAGTAA | 18 |
| E Forward | [Label 2]-GGAAGAGACAGGTACGTTAATAGTTAATAG | 19 |
| E Reverse | [Capture Moiety]-AAAAGAAGGTTTTACAAGACTCACGTTAAC | 20 |

Any capture moieties disclosed or otherwise contemplated herein may be utilized with SEQ
ID NOS: 18 and 20. In the context of this non-limiting example, the capture moiety
utilized was Biosg. Label 1 and Label 2 are each selected from any labels disclosed or
otherwise contemplated herein. In the context of this non-limiting example, Label 1 was
FAM, and Label 2 was DIG.

FIG. 9 depicts a map showing RT-RPA primer sequences in SARS-CoV-2 genomic regions for use in one non-limiting embodiment of an RT-RPA Basic assay constructed in accordance with the present disclosure.

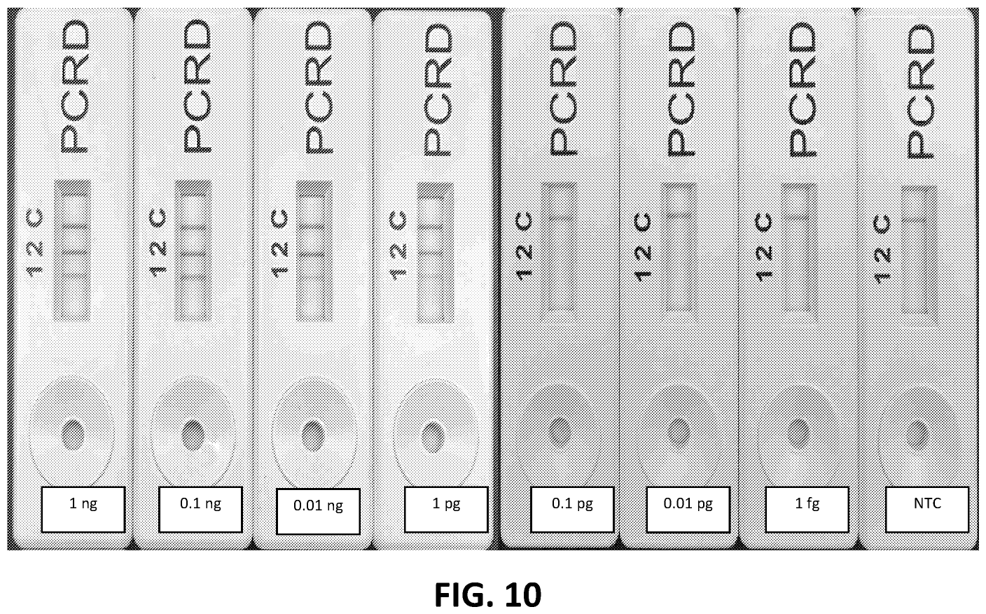

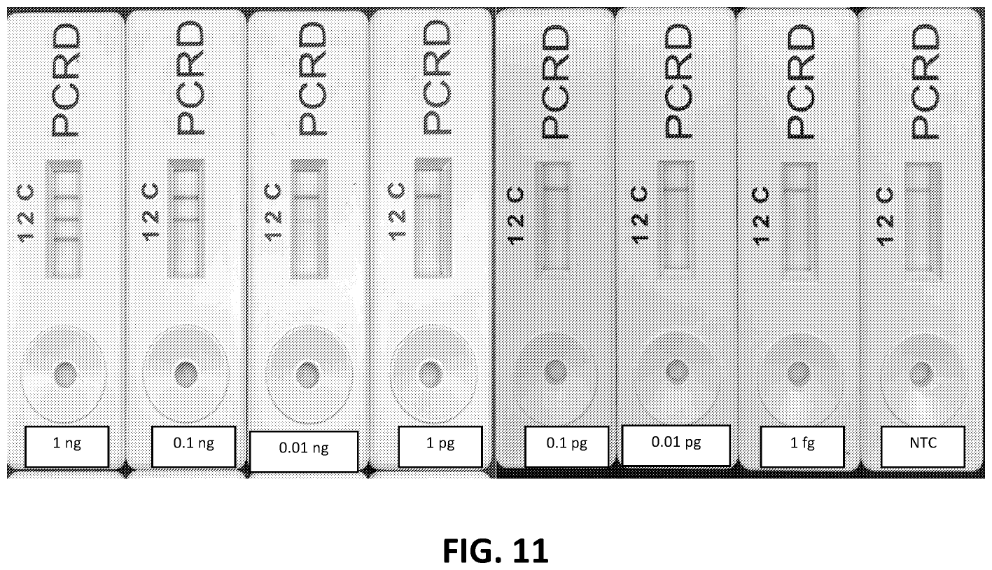

FIGS. 10-11 illustrate a serial dilution sensitivity assay of the RT-RPA Basic assay using Twist Synthetic SARS-CoV-2 RNA Control 2 (Wuhan) diluted in water as the sample. Dilutions correspond to the following amounts of Control 2: from left to right, 1 ng, 0.1 ng, 0.01 ng, 1 pg, 0.1 pg, 0.01 pg, and 1 fg. NTC, negative control. These serial dilution limit of detection assays of the RT-RPA Basic method used plasmid artificial positive control (APC) diluted in water (FIG. 10) or virus transport medium (VTM; FIG. 11) as the sample. As can be seen, the APC could be detected at the lowest concentration tested (1 pg). FIG. 11 demonstrates another limit of detection of the RT-RPA Basic method using Synthetic SARS-CoV-2 RNA Control 2 (Wuhan) diluted in VTM as the sample. As can be seen, SARS-CoV-2 RdRp RNA of the synthetic control was also detected at a concentration as low as 0.01 ng. The SARS-CoV-2 RNA of E was detected at a concentration of 0.1 ng.

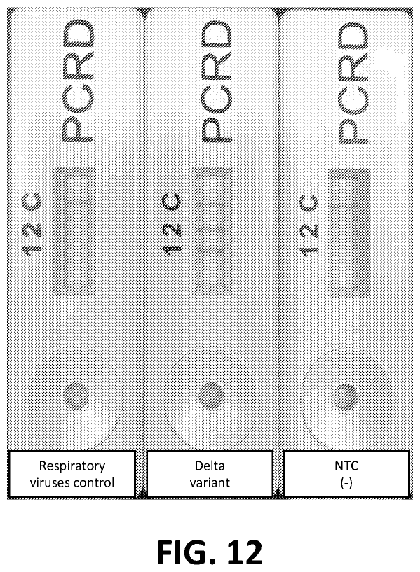

FIG. 12 demonstrates the specificity of the RT-RPA Basic assay. Left, the assay did not amplify (positive reaction) products from either the total respiratory viral panel control (swab) combined with the MERS Coronavirus RNA control. Center, positive reaction of the RdRp and E genes from the synthetic SARS-CoV-2 Delta variant RNA Artificial Positive Control. Right, NTC included as the negative control. The specificity assay demonstrates that the assays disclosed herein can specifically detect variants of SARS-CoV-2 and can also detect other coronaviruses and distinguish between those infections and SARS-CoV-2 infection.

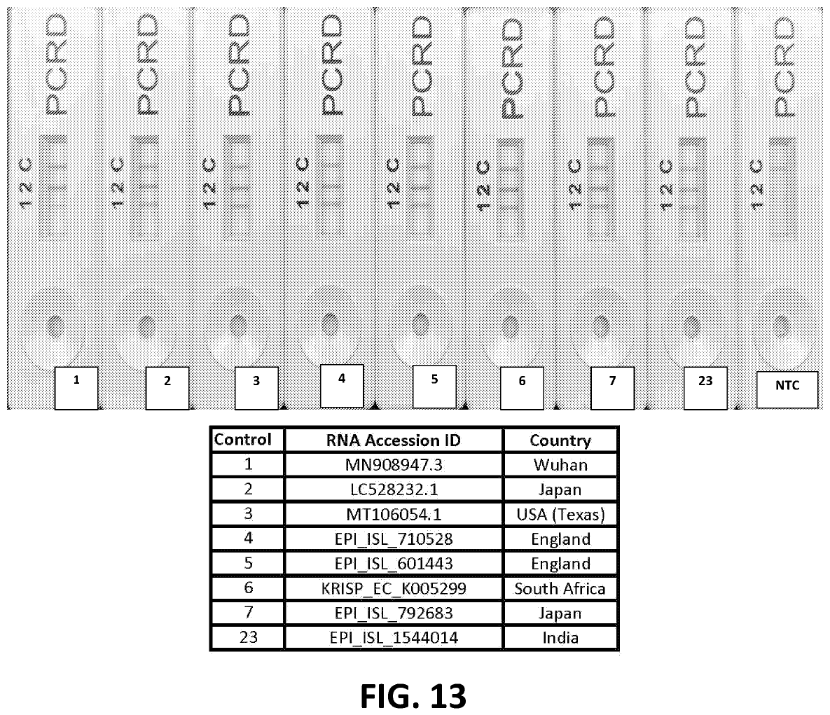

FIG. 13 illustrates a specificity assay of the RT-RPA Basic assay using various synthetic SARS-CoV-2 RNA controls constructed as described herein. Top, broad detection of SARS-CoV-2 variants by RT-RPA Basic using synthetic RNA Artificial Positive Control. NTC is included as the negative control (far right). Bottom, the table shows the identifier reaction numbers corresponding to the accession number of the SARS-CoV-2 variant tested and country of origin. This specificity assay illustrates the broad detection capability of the RT-RPA Basic assay using various synthetic SARS-CoV-2 RNA controls.

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A kit, comprising:
(i) a polymerase amplification oligonucleotide primer pair comprising an isolated forward oligonucleotide primer and an isolated reverse oligonucleotide primer for a portion of a SARS-CoV-2 RNA-dependent RNA polymerase (RdRp) or Helicase (Hel) gene sequence; and
(ii) a polymerase amplification oligonucleotide primer pair comprising an isolated forward oligonucleotide primer and an isolated reverse oligonucleotide primer for a portion of a SARS-CoV-2 Envelope (E) gene sequence; and
wherein the oligonucleotide primers comprise adenine (A), cytosine (C), guanine (G), and thymine (T) residues.

2. The kit of claim 1, wherein each of the reverse oligonucleotide primers is conjugated to a capture moiety.

3. The kit of claim 2, wherein the capture moiety is biotin.

4. The kit of claim 1, further comprising an isolated oligonucleotide probe for the RdRp or Hel gene sequence and an isolated oligonucleotide probe for the E gene sequence, wherein each of the isolated oligonucleotide probes is conjugated to a label, and wherein the two labels are different.

5. The kit of claim 4, wherein:
the forward oligonucleotide primer for the RdRp gene sequence comprises SEQ ID NO:1;
the reverse oligonucleotide primer for the RdRp gene sequence comprises SEQ ID NO:2;
the oligonucleotide probe for the RdRp gene sequence comprises SEQ ID NO:3;
the forward oligonucleotide primer for the E gene sequence comprises SEQ ID NO:4;
the reverse oligonucleotide primer for the E gene sequence comprises SEQ ID NO:5; and
the oligonucleotide probe for the E gene sequence comprises SEQ ID NO:6.

6. The kit of claim 4, wherein:
the forward oligonucleotide primer for the RdRp gene sequence comprises SEQ ID NO:11;
the reverse oligonucleotide primer for the RdRp gene sequence comprises SEQ ID NO:12;
the oligonucleotide probe for the RdRp gene sequence comprises SEQ ID NO:13;
the forward oligonucleotide primer for the E gene sequence comprises SEQ ID NO:14;
the reverse oligonucleotide primer for the E gene sequence comprises SEQ ID NO:15; and
the oligonucleotide probe for the E gene sequence comprises SEQ ID NO:16.

7. The kit of claim 2, wherein each of the isolated forward oligonucleotide primers is conjugated to a label, and wherein the two labels are different.

8. The kit of claim 7, wherein:
the forward oligonucleotide primer for the RdRp gene sequence comprises SEQ ID NO:7;
the reverse oligonucleotide primer for the RdRp gene sequence comprises SEQ ID NO:8;
the forward oligonucleotide primer for the E gene sequence comprises SEQ ID NO:9; and
the reverse oligonucleotide primer for the E gene sequence comprises SEQ ID NO:10.

9. The kit of claim 7, wherein:
the forward oligonucleotide primer for the RdRp gene sequence comprises SEQ ID NO:17;
the reverse oligonucleotide primer for the RdRp gene sequence comprises SEQ ID NO:18;
the forward oligonucleotide primer for the E gene sequence comprises SEQ ID NO:19; and
the reverse oligonucleotide primer for the E gene sequence comprises SEQ ID NO:20.

10. The kit of claim 1, further comprising at least one artificial positive control that contains RdRp and E gene sequences of SARS-CoV-2.

11. The kit of claim 1, further comprising an RPA oligonucleotide pair for at least one positive control.

12. The kit of claim 1, further comprising at least one reaction chamber for performing a polymerase amplification reaction.

13. The kit of claim 12, wherein the polymerase amplification reaction is a recombinase polymerase amplification (RPA) reaction.

14. The kit of claim 12, wherein the polymerase amplification reaction is a PCR reaction.

15. The kit of claim 1, further comprising at least one nucleic acid lateral flow assay device.

16. The kit of claim 1, further comprising at least one collection device.

17. The kit of claim 16, wherein the at least one collection device is an elution independent collection device.

18. The kit of claim 1, further comprising at least one elution independent collection device and at least one nucleic acid lateral flow assay device.

19. The kit of claim 1, further comprising at least one nasopharyngeal swab.

20. A nucleic acid detection system, comprising:
(i) a polymerase amplification oligonucleotide primer pair comprising an isolated forward oligonucleotide primer and an isolated reverse oligonucleotide primer for at least a portion of a SARS-CoV-2 RNA-dependent RNA polymerase (RdRp) or Helicase (Hel) gene sequence, wherein the oligonucleotide primers comprise adenine (A), cytosine (C), guanine (G), and thymine (T) residues;
(ii) a polymerase amplification oligonucleotide primer pair comprising an isolated forward oligonucleotide primer and an isolated reverse oligonucleotide primer for at least a portion of a SARS-CoV-2 Envelope (E) gene sequence, wherein the oligonucleotide primers comprise adenine (A), cytosine (C), guanine (G), and thymine (T) residues;
(iii) at least one collection device;
(iv) at least one reaction chamber for performing a polymerase amplification reaction, wherein the at least one reaction chamber comprises one or more reagents for performing the polymerase amplification reaction; and
(v) at least one nucleic acid lateral flow assay device.

21. The system of claim 20, wherein the at least one collection device is an elution independent collection device.

22. The system of claim 20, wherein the at least one reaction chamber comprises a reagent composition comprising a recombinase, a polymerase, and a single-stranded DNA binding protein.

23. A screening method, comprising:
obtaining a mammalian sample suspected of containing SARS-CoV-2;
combining the mammalian sample with the oligonucleotide primer pairs of the kit of claim 1 and an RPA reagent composition to provide a mixture and incubating the mixture under conditions that allow amplification to occur, wherein the RPA reagent composition comprises a recombinase, a polymerase, and a single-stranded DNA binding protein;
contacting the incubated mixture with a nucleic acid lateral flow assay device;
detecting E RPA products and RdRp or Hel RPA products via the nucleic acid lateral flow assay device;
determining that SARS-CoV-2 is present in the mammalian sample if RdRp/Hel and E RPA products are detected; and
determining that a related coronavirus is present in the mammalian sample if E RPA products are detected and RdRp/Hel RPA products are not detected.

24. A screening method, comprising:
obtaining a mammalian sample suspected of containing SARS-CoV-2;
combining the mammalian sample with the oligonucleotide primer pairs of the kit of claim 1 and a PCR reagent composition to provide a mixture and incubating the mixture under conditions that allow amplification to occur;
contacting the incubated mixture with a nucleic acid lateral flow assay device;
detecting E PCR products and RdRp or Hel PCR products via the nucleic acid lateral flow assay device;
determining that SARS-CoV-2 is present in the mammalian sample if RdRp/Hel and E PCR products are detected; and
determining that a related coronavirus is present in the mammalian sample if E PCR products are detected and RdRp/Hel PCR products are not detected.


SEQUENCE LISTING (24 sequences)

SEQ ID NO: 1 - DNA, 35 bp, Artificial sequence
  Forward oligo primer for SARS-CoV-2 RdRp gene sequence
tggaacctcatcaggagatgccacaactgcttatg

SEQ ID NO: 2 - DNA, 35 bp, Artificial sequence
  Reverse oligo primer for SARS-CoV-2 RdRp gene sequence
gagacactcataaagtctgtgttgtaaattgcgga

SEQ ID NO: 3 - DNA, 47 bp, Artificial Sequence
  Probe for SARS-CoV-2 RdRp gene sequence
gtgtttttaacatttgtcaagctgtcacggccatgttaatgcacttt

SEQ ID NO: 4 - DNA, 35 bp, Artificial Sequence
  Forward oligo primer for SARS-CoV-2 E gene sequence
gtactcattcgtttcggaagagacaggtacgttaa

SEQ ID NO: 5 - DNA, 32 bp, Artificial Sequence
  Reverse oligo primer for SARS-CoV-2 E gene sequence
aaacgtaaaaagaaggttttacaagactcacg

SEQ ID NO: 6 - DNA, 54 bp, Artificial Sequence
  Probe for SARS-CoV-2 E gene sequence
tttcgtggtattcttgctagttacactagccatccttatgcgcttcgattgtgt

SEQ ID NO: 7 - DNA, 35 bp, Artificial Sequence
  Forward oligo primer for SARS-CoV-2 Hel gene sequence
cataaattagtcttgtctgttaatccgtatgtttg

SEQ ID NO: 8 - DNA, 35 bp, Artificial Sequence
  Reverse oligo primer for SARS-CoV-2 Hel gene sequence
gtgatttacaataatagctcatacctcctaagtaa

SEQ ID NO: 9 - DNA, 30 bp, Artificial Sequence
  Forward oligo primer for SARS-CoV-2 E gene sequence
ggaagagacaggtacgttaatagttaatag

SEQ ID NO: 10 - DNA, 30 bp, Artificial Sequence
  Reverse oligo primer for SARS-CoV-2 E gene sequence
aaaagaaggttttacaagactcacgttaac

SEQ ID NO: 11 - DNA, 35 bp, Artificial Sequence; identical to SEQ ID NO: 1
  Forward oligo primer for SARS-CoV-2 RdRp gene sequence

SEQ ID NO: 12 - DNA, 35 bp, Artificial Sequence; identical to SEQ ID NO: 2
  Reverse oligo primer for SARS-CoV-2 RdRp gene sequence
  misc_feature (1)..(1): Capture moiety attached to 5′ end

SEQ ID NO: 13 - DNA, 47 bp, Artificial Sequence; identical to SEQ ID NO: 3
  Oligo probe for SARS-CoV-2 RdRp gene sequence
  misc_feature (1)..(1): Label attached to 5′ end of primer
  misc_feature (33)..(33): THF
  misc_feature (47)..(47): C3 spacer attached to 3′ end of primer

SEQ ID NO: 14 - DNA, 35 bp, Artificial Sequence; identical to SEQ ID NO: 4
  Forward oligo primer for SARS-CoV-2 E gene sequence

SEQ ID NO: 15 - DNA, 32 bp, Artificial Sequence; identical to SEQ ID NO: 5
  Reverse oligo primer for SARS-CoV-2 E gene sequence
  misc_feature (1)..(1): Capture moiety attached to 5′ end of primer

SEQ ID NO: 16 - DNA, 54 bp, Artificial Sequence; identical to SEQ ID NO: 6
  Oligo probe for SARS-CoV-2 E gene sequence
  misc_feature (1)..(1): Label attached to 5′ end of primer
  misc_feature (38)..(38): THF
  misc_feature (54)..(54): C3 spacer attached to 3′ end of primer

SEQ ID NO: 17 - DNA, 35 bp, Artificial Sequence; identical to SEQ ID NO: 7
  Forward oligo primer for SARS-CoV-2 Hel gene sequence
  misc_feature (1)..(1): Label attached to 5′ end of primer

SEQ ID NO: 18 - DNA, 35 bp, Artificial Sequence; identical to SEQ ID NO: 8
  Reverse oligo primer for SARS-CoV-2 Hel gene sequence
  misc_feature (1)..(1): Capture moiety attached to 5′ end of primer

SEQ ID NO: 19 - DNA, 30 bp, Artificial Sequence; identical to SEQ ID NO: 9
  Forward oligo primer for SARS-CoV-2 E gene sequence
  misc_feature (1)..(1): Label attached to 5′ end of primer

SEQ ID NO: 20 - DNA, 30 bp, Artificial Sequence; identical to SEQ ID NO: 10
  Reverse oligo primer for SARS-CoV-2 E gene sequence
  misc_feature: Capture moiety attached to the 5′ end of the primer

SEQ ID NO: 21 - RNA, 224 nt, SARS-CoV-2
gugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuaua

SEQ ID NO: 22 - RNA, 201 nt, SARS-CoV-2
ugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucgugu

SEQ ID NO: 23 - RNA, 154 nt, SARS-CoV-2
cgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuucca

SEQ ID NO: 24 - RNA, 154 nt, SARS-CoV-2
ucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuu